(19) United States
(12) Patent Application Publication
(10) Pub. No.: US 2023/0060057 A1
(43) Pub. Date: Feb. 23, 2023

(54) FIXATOR APPARATUS FOR THE TREATMENT OF OPEN CALCANEAL FRACTURES

(71) Applicant: ATATÜRK ÜNIVERSITESI BILIMSEL ARASTIRMA PROJELERI BIRIMI, Yakutiye, Erzurum (TR)

(72) Inventors: Kutsi TUNCER, Yakutiye, Erzurum (TR); Ahmet Emre PAKSOY, Palandöken, Erzurum (TR)

(21) Appl. No.: 17/760,175

(22) PCT Filed: Sep. 15, 2021
(86) PCT No.: PCT/TR2021/050935

§ 371 Date: Aug. 4, 2022

(30) Foreign Application Priority Data
Sep. 22, 2020 (TR) 2020/15042

(51) Int. Cl. A61B 17/56

(52) U.S. Cl. CPC A61B 17/56; A61B 17/66

(57) ABSTRACT

A fixator apparatus has a lateral fixator distal apparatus, a lateral fixator proximal apparatus, a lateral fixator joint, a posterior joint, an Essex-lopresti apparatus, a lateral transmission housing, a posterior transmission housing, and a distraction apparatus. The fixator apparatus facilitates reduction, provides two-plan fixation, and increases stability.

### TECHNICAL FIELD

The invention relates to a fixator apparatus for use in the treatment of open calcaneal fractures in the field of orthopedics.

The invention particularly relates to a fixator apparatus to be used in the treatment of open calcaneal fractures in the field of orthopedics, facilitating reduction and increasing stability by providing two-plan fixation.

### PRESENT STATE OF THE ART

Detecting calcaneal fractures is difficult in the field of orthopedics and traumatology. Plaster applications are risky for fragmented, open or edematous fractures extending to the joint. Plate applications are difficult and problematic regarding the comminuted fractures. In addition, in the event that the fracture in question is an incision in the skin, known as an open fracture, placing a plate is contraindicated due to infection. In the technique called Essex-lopresti, in which the fracture ends are joined by percutaneously entering the posterior calcaneus and using a screw or shank nail as a joystick, stability is not sufficient and must be supported with plaster. Plaster application is not preferred for the open fractures since wound complications will occur by wrapping plaster on the fracture. The external fixator is a single plane apparatus applied on the skin that is used for open fractures. Reduction difficulties and stability deficiencies may occur.

The American patent application document numbered US202016735752 was determined in the patent and literature search conducted for the present state of the art. In the said document, the external fixator configuration and the method for the said construction are explained. According to the document, the relevant invention consists of a main body for connecting multiple pins positioned on opposite sides of a broken bone with one more rod, multiple clamp assemblies configured to clamp onto rods, a clamping device for adjusting the location of a clamp assembly along the length of its rod. There is no explanation in the document regarding the facilitation of the reduction.

In conclusion, there are many problems and negativities in the technical field in question, as mentioned above, and existing applications are insufficient in solving these problems and negativities. This necessitates a development and an innovation in the present art.

### BRIEF DESCRIPTION OF THE INVENTION

The present invention relates to a fixator apparatus that meets the above-mentioned needs and eliminates all the disadvantages and brings additional advantages, that is used for open calcaneal fractures.

The primary object of the invention is to achieve a fixator apparatus to be used on the open calcaneal fractures. Fixator apparatus according to the invention facilitates reduction, provides two-plan fixation, and increases stability. External fixation is applied only in the lateral plan in the old-style fixators that are included in the state of the art; however, the posterior plane, which is far from the neurovascular structures, is a safe area to contribute to the stability in the apparatus according to the invention.

Another object of the invention is to provide a fixation device that prevents wound care difficulties and minimizes the risk of infection in the open wound problem.

Structural features and the characteristics and all the advantages of the invention will be understood more clearly with the detailed description and figures below. For this reason, the evaluation should be made taking into account the relevant detailed description.

### DESCRIPTION OF THE FIGURES FOR A BETTER UNDERSTANDING OF THE INVENTION

FIG. 1 provides the top view of the fixator apparatus according to the invention.

FIG. 2 provides the 3-D overview of the fixator apparatus according to the invention on a lateral plane.

FIG. 3 provides the top view of the fixator apparatus according to the invention during the application.

### DESCRIPTION OF THE REFERENCE

F Fixator apparatus
1 Lateral fixator distal apparatus
2 Lateral fixator proximal apparatus
3 Lateral fixator joint
4 Posterior joint
5 Essex-lopresti apparatus
6 Lateral transmission housing
7 Posterior transmission housing
8 Distraction apparatus

### DETAILED DESCRIPTION OF THE INVENTION

In this detailed description, preferred embodiments of the fixator apparatus (F) of the invention are explained only for a better understanding of the relevant subject and without any limiting effect.

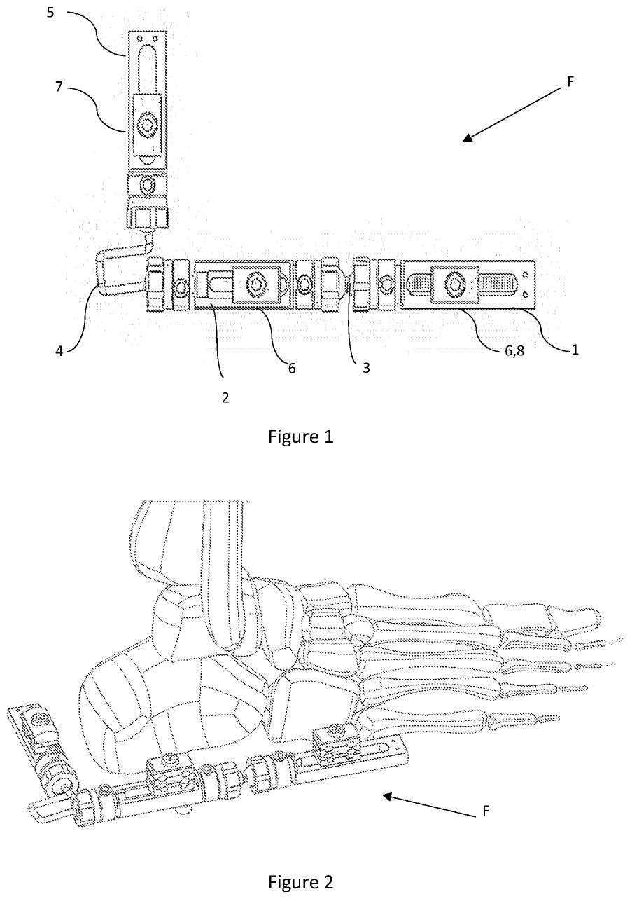

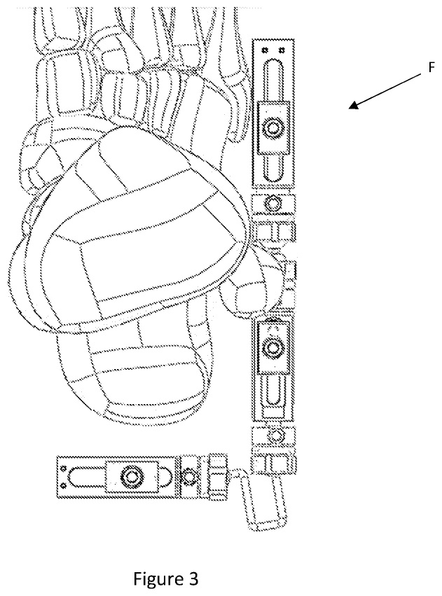

The invention relates to a fixator apparatus (F) for use in the treatment of open calcaneal fractures. FIG. 1 provides the top view of the fixator apparatus (F) according to the invention. Accordingly, said fixator apparatus (F) consists lateral transmission housings (6) in the form of threaded wire with a thickness preferably varying between 2-2.5 mm, in order to enter the posterior part of the calcaneus and the cuboid bone. The embodiment of the fixator apparatus (F) according to the invention consists of at least one lateral fixator distal apparatus (1) to hold the lateral transmission housings (6) to be fixed to the cuboid bone, at least one lateral fixator proximal apparatus (2) to hold the lateral transmission housings (6) that will hold the lateral transmission to be fixed to the posterior part of the calcaneus. In addition, the embodiment of the invention consists of at least one lateral fixator joint (3) to facilitate reduction by allowing angulation between the lateral fixator distal apparatus (1) and the lateral fixator proximal apparatus (2).

The embodiment of the fixator apparatus (F) according to the invention consists of at least one essex-lopresti apparatus (5) to fix the posterior transmission housing (7), which will hold the thick transmission housing received from the posterior. Additionally, the embodiment of the fixator apparatus (F) consists of at least one posterior joint (4) that facilitates reduction by allowing angulation between the essex-lopresti apparatus (5) in the background and the fixator in the lateral plane (lateral fixator distal apparatus (1), lateral fixator proximal apparatus (2), and lateral fixator joint (3)).

Fixator apparatus (F) also consists of a posterior transmission housing (7) in addition to the said lateral transmission housings (6). Said posterior transmission housing (7) will hold threaded transmissions, the thickness of which preferably varies between 4-5 mm, in such a way that it enters the calcaneus from posterior to anterior.

In the invention, at least one distraction apparatus (8) is configured to provide the opportunity to perform compression—distraction by opening and closing the lateral apparatus (lateral fixator distal apparatus (1) and lateral fixator proximal apparatus (2)).

During the application of the relevant fixator apparatus (F), lateral transmission housings (6) are fixated to cuboid bone and calcaneus posterior and lateral fixator distal apparatus (1) and lateral fixator proximal apparatus (2). Then, the reduction is brought to the desired level by using the lateral fixator joint (3) and the posterior joint (4). The threaded part of the said posterior transmission housing (7) is fixed from the posterior calcaneus posterior to the distal fracture with the posterior transmission housing (7). Essex-lopresti apparatus (5) is mounted on the said posterior transmission housing (7), ensuring the fixation of the posterior transmission housing (7). In the next step, the fracture line is brought closer to or moved away from one another by using the distraction apparatus (8).

Present invention is a fixator apparatus (F) to be used on the open calcaneal fractions with the aim of solving technical problems and achieving all the advantages set forth on the detailed description, and consists of
Lateral transmission housing (6), which will hold more than one lateral transmission in the form of a threaded wire, to enter the posterior part of the calcaneus and the cuboid bone,
At least one lateral fixator distal apparatus (1) to hold the lateral transmission housings (6) to be fixed to the cuboid bone,
At least one lateral fixator proximal apparatus (2) to hold the lateral transmission housings (6) to be fixed to the posterior part of the calcaneus,
At least one lateral fixator joint (3) to facilitate reduction by allowing angulation between the lateral fixator distal apparatus (1) and the lateral fixator proximal apparatus (2),
At least one essex-lopresti apparatus (5) to fix the posterior transmission housing (7), which will hold the thick transmission housing received from the posterior,
At least one posterior joint (4) that facilitates reduction by allowing the angulation between the aforementioned essex-lopresti apparatus (5) and the fixator in the lateral plane,
A posterior transmission housing (7) that will hold the posterior transmission in the form of a threaded wire with the aim of entering the calcaneus from posterior to anterior.

1. A fixator apparatus for use in the treatment of open calcaneal fractures, the fixator apparatus comprising:
a lateral transmission housing, which will hold more than one lateral transmission in the form of a threaded wire, to enter the posterior part of the calcaneus and the cuboid bone,
at least one lateral fixator distal apparatus to hold the lateral transmission housings to be fixed to the cuboid bone,
at least one lateral fixator proximal apparatus to hold the lateral transmission housings to be fixed to the posterior part of the calcaneus,
at least one lateral fixator joint to facilitate reduction by allowing angulation between the lateral fixator distal apparatus and the lateral fixator proximal apparatus,
at least one essex-lopresti apparatus to fix the posterior transmission housing, which will hold the thick transmission housing received from the posterior,
at least one posterior joint that facilitates reduction by allowing the angulation between the aforementioned essex-lopresti apparatus and the fixator in the lateral plane, and
a posterior transmission housing that will hold the posterior transmission in the form of a threaded wire with the aim of entering the calcaneus from posterior to anterior.

2. A fixator apparatus according to claim 1, comprising at least one distraction apparatus that enables compression and distraction by opening and closing the lateral apparatus.

3. A fixator apparatus according to claim 1, wherein the lateral transmission housings to hold lateral transmissions have thicknesses ranging from 2-2.5 mm.

4. A fixator apparatus according to claim 1, wherein the posterior transmission housing to hold the posterior transmission have thicknesses between 4-5 mm.
